(19) United States
(12) Patent Application Publication
(10) Pub. No.: US 2017/0240958 A1
(43) Pub. Date: Aug. 24, 2017

(54) BIOMARKER OF MYCOBACTERIOSIS OR MYCOBACTERIAL INFECTION

(71) Applicants: KEIO UNIVERSITY, Tokyo (JP); NIKON CORPORATION, Tokyo (JP)

(72) Inventors: Naoki HASEGAWA, Tokyo (JP); Tomoyasu NISHIMURA, Yokohama-shi (JP)

(21) Appl. No.: 15/433,735

(22) Filed: Feb. 15, 2017

(30) Foreign Application Priority Data
Feb. 19, 2016 (JP) 2016-030020

(51) Int. Cl. C12Q 1/68

(52) U.S. Cl. CPC C12Q 1/689; C12Q 2600/158; C12Q 2600/178

(57) ABSTRACT

The present invention provides a solid carrier capable of easily and accurately evaluating the activity of mycobacteriosis or the presence or absence of mycobacterial infection. The solid carrier for detecting mycobacteriosis or mycobacterial infection includes at least one probe including a polynucleotide consisting of a base sequence complementary to all or a part of a base sequence having 85% or more identity with respect to a base sequence set forth in SEQ ID NO: 1, which is immobilized on a surface thereof.

### BACKGROUND OF THE INVENTION

Field of the Invention

The present invention relates to a biomarker of mycobacteriosis or mycobacterial infection.

Description of Related Art

Examples of clinically problematic mycobacteriosis include tuberculosis, leprosy, non-tuberculous mycobacterial infection (NTM) and the like. Although tuberculosis and leprosy have became curable infections, the pathology and effective treatment methods for NTM have not been defined yet. According to the National Survey of the Ministry of Health, Labor and Welfare in 2014, the estimated prevalence of NTM diseases was 14.7 people out of every 100,000 people, and a newly recorded annualized prevalence of tuberculosis was over 12.9 people out of every 100,000 people, and therefore, it is considered that NTM diseases will become more problematic as an intractable infection in the future.

Mycobacterium avium complex (MAC) pulmonary diseases account for approximately 90% of NTM diseases in Japan. Although the causative pathogen MAC is ubiquitous in the living environment such as water or soil, it does not infect from person to person, and except for the highly characteristic tendency for MAC disease to be frequently found in middle-aged and elderly women who do not have a smoking history and underlying diseases, the pathogenesis of MAC disease has not been clarified. As a treatment method, for example, although a combination antibacterial chemotherapy may be carried out, since there is no criteria for determining the starting time of the treatment, the treatment duration and the time for terminating the treatment, a recurrence or relapse of the disease is frequent. In addition, there are cases in which follow-up observation is possible for a long time without treatment, and cases in which a disease condition suddenly worsens to death due to treatment resistance. However, at present, since there is no biomarker for evaluating the activity of MAC pulmonary disease, it is very difficult to evaluate the activity of MAC pulmonary disease and predict the prognosis thereof.

In recent years, microRNA (miRNA) has attracted attention as a biomarker of various diseases. miRNA is known to perform down-regulation of gene expression by various methods such as the repression of translation of non-coding 21 to 25 bp RNA, mRNA degradation, deadenylation, and so on. Since it was first suggested in 2002 that miRNA is related to disease, research has progressed particularly in the tumor area, indicating that miRNA can be used as an indicator of carcinogenesis. In 2007, it was reported that miRNA and mRNA are present in exosomes, and the exosomes can be used in intracellular transport of miRNA. Furthermore, in 2008, specifically, it was suggested that miRNA is used as a biomarker by measuring tumor-specific miRNA released into blood from diseased tissue, and is receiving much attention for being applied to less invasive test methods.

In Japanese Translation of PCT International Application Publication No. 2013-502931, it is disclosed that miRNA can be used as a serum biomarker or plasma biomarker for characterizing a pulmonary disease of a patient.

### SUMMARY OF THE INVENTION

One aspect of the present invention is to provide a solid carrier for detecting mycobacteriosis or mycobacterial infection, which has at least one probe including a polynucleotide consisting of a base sequence complementary to all or a part of a base sequence having 85% or more identity with respect to a base sequence set forth in SEQ ID NO: 1, which is immobilized on a surface of the carrier.

Another aspect of the present invention is to provide a method for evaluating the activity of mycobacteriosis or the presence or absence of mycobacterial infection, the method including: bringing a sample derived from a subject into contact with a solid carrier for detecting mycobacteriosis or mycobacterial infection in which at least one probe includes a polynucleotide consisting of a base sequence complementary to all or a part of a base sequence having 85% or more identity with respect to a base sequence set forth in SEQ ID NO: 1, which is immobilized on a surface of the carrier; and detecting a nucleic acid hybridized with the probe.

Still another aspect of the present invention is to provide a kit for detecting mycobacteriosis or mycobacterial infection, which includes: a reverse transcription primer including a base sequence complementary to a partial sequence at the 3′ end of a base sequence having 85% or more identity with respect to a base sequence set forth in SEQ ID NO: 1; forward and reverse primers for amplifying a reverse transcription product that is obtained by reverse transcription using miRNA consisting of a base sequence having 85% or more identity with respect to the base sequence set forth in SEQ ID NO: 1 and the reverse transcription primer; and a labeled oligonucleotide probe including a sequence complementary to a part of the resulting amplification product obtained by the forward and reverse primers.

### BRIEF DESCRIPTION OF THE DRAWINGS

FIG. 1A is a graph showing the result of measuring the concentration of hsa-miR-346 in serum for a patient with MAC disease and a healthy subject according to Example 1.

FIG. 1B is a graph showing the result of measuring the concentration of hsa-miR-532-3p in serum for a patient with MAC disease and a healthy subject according to Example 1.

FIG. 1C is a graph showing the result of measuring the concentration of hsa-miR-3155b in serum for a patient with MAC disease and a healthy subject according to Example 1.

FIG. 1D shows a graph of the result of measuring the concentration of hsa-miR-4446-3p in serum for a patient with MAC disease and a healthy subject according to Example 1.

FIG. 1E is a graph showing the result of measuring the concentration of hsa-miR-7515 in serum for a patient with MAC disease and a healthy subject according to Example 1.

FIG. 2 is a graph showing the result of measuring the concentration of hsa-miR-346 in serum for a tuberculosis patient and a healthy subject according to Example 2.

FIG. 3 is a graph showing the result of measuring the concentration of hsa-miR-346 in the supernatant of macrophages for a patient with MAC disease and a healthy subject according to Example 3.

FIG. 4A is a graph showing the result of sputum culture and the change in hsa-miR-346 concentration in serum over time for a patient with MAC disease after treatment initiated by the administration of rifampicin (RIF), ethambutol (EMB), and clarithromycin (CLR) according to Example 4.

FIG. 4B is a graph showing the result of sputum culture and the change in hsa-miR-346 concentration in serum over time for a patient with MAC disease after treatment initiated by the administration of rifampicin (RIF), ethambutol (EMB), clarithromycin (CLR), and amikacin (AMK) according to Example 4.

### DETAILED DESCRIPTION OF THE INVENTION

<<Biomarker of Mycobacteriosis or Mycobacterial Infection>>

In one embodiment, the present invention provides a biomarker of mycobacteriosis or mycobacterial infection, which is microRNA (miRNA) consisting of a base sequence having 85% or more identity with respect to a base sequence set forth in SEQ ID NO: 1.

As a result of a course of research, the inventors have focused on the presence of miRNA which is highly expressed in the serum of a mycobacteriosis patient, and have found that miR-346 is deeply involved in mycobacteriosis, and thus completed the present invention.

According to the biomarker of mycobacteriosis or mycobacterial infection in the embodiment, the activity of mycobacteriosis or the presence or absence of mycobacterial infection can be easily and accurately evaluated.

The term “mycobacteriosis” generally used herein refers to an infection caused by bacteria belonging to the genus Mycobacterium classified as gram-positive bacteria and including tubercular bacilli. Examples of mycobacteriosis include tuberculosis, leprosy, and non-tuberculous mycobacterial infection (NTM) diseases. Further, examples of NTM diseases include Mycobacterium avium complex (MAC) disease in which there is no distinction between the two bacterial species of Mycobacterium avium and Mycobacterium intracellulare, and infection caused by Mycobacterium kansasii. These three species cause over 91% of NTM diseases.

<miRNA>

The biomarker of mycobacteriosis or mycobacterial infection of the embodiment is miRNA consisting of a base sequence having 85% or more identity with respect to the base sequence set forth in SEQ ID NO: 1, contained in samples taken from humans (including biosamples such as blood, saliva, tears, sweat, urine, and so on). Measuring the expression level of the miRNA may be used to evaluate the activity of mycobacteriosis or the presence or absence of mycobacterial infection.

The term “miRNA” generally used herein refers to that which is called mature miRNA. Mature miRNA is endogenous non-coding RNA containing approximately 20 to 25 bases, which is encoded on a genome. miRNA is transcribed into a primary transcript (primary miRNA, hereinafter, referred to as “Pri-miRNA”) containing several hundreds to thousands of bases from miRNA genes on genome DNA, and then processed to be precursor miRNA (pre-miRNA) having an approximately 60 to 70 base-long hairpin structure. After that, the pre-miRNA is transferred into the cytoplasm from the nucleus and then processed, resulting in an approximately 20 to 25 base-long double-stranded mature miRNA. It is known that one strand of the double-stranded mature miRNA creates a complex with a protein called RISC, acting on target gene mRNA so as to repress the translation of the target gene.

The miRNA of the embodiment is miRNA consisting of a base sequence having 85% or more, for example, 90% or more, for example, 95% or more, for example, 99% or more, or, for example, 100% identity with respect to the base sequence set forth in SEQ ID NO: 1 (5′-UGUCUGCCCGCAUGCCUGCCUCU-3′). An example of the miRNA of the embodiment is miRNA having 100% identity with respect to the base sequence set forth in SEQ ID NO: 1, that is, miR-346.

The miRNA consisting of a base sequence having 85% or more identity with respect to the base sequence set forth in SEQ ID NO: 1 also includes miRNA consisting of a base sequence in which 1 to 3 bases are added, substituted or deleted with respect the base sequence set forth in SEQ ID NO: 1.

<<Method for Evaluating the Activity of Mycobacteriosis or the Presence or Absence of Mycobacterial Infection for Subject>>

In one embodiment, the present invention provides a method for evaluating the activity of mycobacteriosis in a subject, the method including measuring the concentration of the above-described biomarker in a sample obtained from the subject.

According to the evaluation method of the embodiment, the activity of mycobacteriosis or the presence or absence of mycobacterial infection, and a degree thereof can be easily and accurately evaluated. The evaluation method of the embodiment can also be used for evaluation of the therapeutic effect for patients who are suffering from mycobacteriosis and receiving treatment. Based on this evaluation result, a treatment plan for mycobacteriosis can be clearly determined.

The term “disease activity” used herein is a term used to generally express the progress of a disease, symptoms, and the degree of dysfunction in a patient and so on. In the specification, this term specifically refers to the progress of mycobacteriosis, the presence or degree of infection, the severity of symptoms, and so on. In the specification, the evaluation of the disease activity also includes prediction of the prognosis for mycobacteriosis, evaluation of deterioration and improvement of a disease condition, and evaluation of a therapeutic effect.

<Measuring Process>

The evaluation method of the embodiment will be described in detail below.

First, a concentration of the above-described biomarker in a sample obtained from a subject is measured. As the sample obtained from a subject, the above-described <miRNA> may be used. For example, a blood sample can be used. Examples of the blood sample include blood, serum, and plasma. In addition, examples of the subject include a patient with mycobacteriosis, a patient suffering from and receiving treatment for mycobacteriosis, a person suspected of having mycobacteriosis, a person who is at risk of developing mycobacteriosis (e.g., a person who has had mycobacteriosis previously and was diagnosed as cured or the like), and a person suspected of mycobacterial infection.

The miRNA contained in the sample can be extracted and purified by a method known in the art or using a commercially available miRNA kit. In addition, the miRNA expression level can be measured by, for example, a microarray or quantitative RT-PCR.

Next, the measured miRNA expression level is compared with that of a healthy subject. The expression level of the miRNA in serum of the sample obtained from a healthy subject can be measured in advance, or can be measured together with the expression level of the miRNA in a sample obtained from a subject. When using a microarray, the miRNA expression levels can be compared by standardization with total RNA, or when using quantitative RT-PCR, standardization based on an intrinsic control. For an intrinsic control, for example, miR-16-5p or miR-39-3p can be used. Here, a healthy subject may be a person who is not infected by mycobacteria and of the same or different sex, compared to the subject. Multiple healthy subjects can participate in the investigation. The expression level of miRNA to be compared to that of the subject may be expressed by a range. The range of the miRNA expression levels compared to that of the subject may be, but not limited to, for example, (i) a range from a value obtained by subtracting a standard deviation from an average value of a plurality of values measured for each healthy subject to a value obtained by adding the standard deviation to the average value, and (ii) a range from the lower limit to the upper limit of the average value. When the expression level of a healthy subject is in the above range, for example, if the expression level of a subject is higher than the upper limit, it is considered to be higher than that of a healthy subject, and if it is lower than the lower limit, it is considered to be lower than that of a healthy subject.

In the measuring process, as a result of comparing the expression level of miRNA in a sample obtained from a subject with that of miRNA in a sample obtained from a healthy subject, when the expression level of miRNA in the sample obtained from the subject is higher than that of miRNA in the sample obtained from a healthy subject, it can be considered that the above-mentioned subject has a high chance of having mycobacteriosis or to be infected by mycobacteria. In contrast, when the expression level of miRNA in the sample obtained from the subject is similar to or lower than that of miRNA in the sample obtained from a healthy subject, it can be considered that the above-mentioned subject has a high chance of not having mycobacteriosis or not being infected with mycobacteria.

In addition, in the measuring process, the expression level of miRNA in the sample obtained from the subject may be compared with a predetermined specific value. In this case, for example, when the expression level of miRNA in the sample obtained from the subject is higher than the predetermined value, it can be considered that the above-mentioned subject has a high chance of having mycobacteriosis or being infected by mycobacteria, and when the expression level of miRNA in the sample obtained from the subject is lower than the predetermined value, it is considered that the above-mentioned subject has a high chance of not having mycobacteriosis or not being infected by mycobacteria. The predetermined value can be suitably determined by those of ordinary skill in the art by measuring the expression levels of miRNA in the samples obtained from a plurality of mycobacteriosis patients and healthy subjects and examining the distribution thereof.

In addition, in the measuring process, the expression level of miRNA in the sample obtained from the subject may be compared with those of miRNA in the sample obtained from the subject before or after a certain period of time. In this case, for example, when the expression level of miRNA has increased after a certain period of time, it can be considered that a disease condition has degraded, and when the expression level of miRNA has decreased after a certain period of time, it can be considered that a disease condition has improved. According to the above-described method, the presence and degree of a therapeutic effect can be evaluated.

According to the evaluation method of the embodiment, in combination with conventional evaluation methods, the activity of mycobacteriosis may be evaluated.

<<Solid Carrier for Detecting Mycobacteriosis or Mycobacterial Infection>>

In one embodiment, the present invention provides a solid carrier for detecting mycobacteriosis or mycobacterial infection, which has at least one probe including a polynucleotide consisting of a base sequence complementary with a base sequence having 85% or more identity with respect to the base sequence set forth in SEQ ID NO: 1, which is immobilized on a surface of the carrier.

The solid carrier for detecting mycobacteriosis or mycobacterial infection of the embodiment can be used to measure the above-described miRNA by a method using a microarray. By using the solid carrier for detecting mycobacteriosis or mycobacterial infection of the embodiment, the activity of mycobacteriosis or the presence or absence of mycobacterial infection can be easily and accurately evaluated.

The probe used in the solid carrier for detecting mycobacteriosis or mycobacterial infection of the embodiment includes a polynucleotide consisting of a base sequence complementary to all or a part of the above-mentioned miRNA. When the probe has a base sequence complementary to a part of the above-described miRNA, as long as the miRNA can be specifically captured, the length of the “part” (e.g., the number of bases) with respect to the length of the miRNA is not particularly limited, and the length of the “part” may be, for example, 50% or more, 60% or more, 70% or more, 80% or more, 90% or more, or 95% or more the length of the miRNA. From the viewpoint of accurately detecting miRNA, the probe may further contain a sequence not complementary to the miRNA.

The term “polynucleotide” generally used herein includes a plurality of nucleosides linked to each other by a linker moiety such as phosphate, and is usually a configuration in which several nucleotides are linked to each other. Here, the number of nucleosides is not particularly limited. Therefore, for example, the generally termed “oligonucleotide” in which 10 or less nucleosides or nucleotides are linked is also referred to as a “polynucleotide” throughout the specification.

Since the probe of the embodiment requires a molecular degree of freedom, to hybridize with miRNA, it may have a spacer at the end which binds to the solid carrier.

The length of the spacer (e.g., refers to the number of bases) may be, but is not particularly limited to, for example, 3 to 50 bases, for example, 5 to 25 bases. However, a base used for the spacer can be substituted with a linker such as PEG or the like, which has the same length and flexibility as the spacer. In such a case, the number of bases used for the spacer may be 0.

The sample including miRNA used for detection with the solid carrier for detecting mycobacteriosis or mycobacterial infection of the embodiment is not particularly limited, but may be, for example, those used in the above-described <measuring process> when being used in the above-described <<method for evaluating the activity of mycobacteriosis or the presence or absence of mycobacterial infection in a subject>>.

In addition, since the miRNA content in the sample is very small as approximately 0.01 mass %, an miRNA concentrate obtained by fractionation from total RNA extracted from the sample may be used as a sample, or a sample which is not fractionated may be used as a sample.

In one aspect, the solid carrier for detecting mycobacteriosis or mycobacterial infection of the embodiment may be a substrate for detecting mycobacteriosis. As a substrate for immobilizing a probe, which is used in the substrate for detecting mycobacteriosis or mycobacterial infection, for example, a glass substrate, a silicon substrate, a plastic substrate, a metal substrate or the like may be used. As the method for immobilizing a probe on a substrate, for example, a method for immobilizing a probe on a substrate at a high density through photo-lithography or a method for immobilizing a probe on a glass substrate by spotting may be used.

In the case of using photo-lithography, a probe may be synthesized on a substrate.

When a probe is immobilized by spotting, a solid binding site can be provided on the probe, and a solid binding site-recognition site can be provided on the substrate.

As such a combination of solid binding site/solid binding site-recognition site, a combination of a solid binding site provided by modifying the probe with a functional group such as an amino group, a formyl group, an SH group or a succinimidyl ester group, and a solid binding site-recognition site provided by surface-treating a substrate with a silane coupling agent such as an amino group, a formyl group, an epoxy group or a maleimide group, a combination using a gold-thiol linkage or the like may be used.

In addition, as another method for immobilizing a probe by spotting, for example, a method for ejecting, aligning and covalently bonding a probe having a silanol group on a glass substrate or the like may be used.

In another aspect, examples of the solid carrier for detecting mycobacteriosis or mycobacterial infection of the embodiment include beads made of a metal, glass, and a resin, and various membranes, and for such solid carriers, probes can be immobilized by a known method.

A polynucleotide constituting the probe in the embodiment may be any polynucleotide that can hybridize with miRNA, and there is no limitation to the polynucleotide. The polynucleotide may be RNA, DNA, or an artificial nucleic acid such as 2′-O-methyl RNA, locked nucleic acid (LNA), bridged nucleic acid (BNA) or the like.

The term “hybridize” used herein refers to a state where at least a part of miRNA is hybridized with the probe under stringent conditions to form a complementary complex. In addition, the term “stringent conditions” refers to conditions described in Molecular Cloning—A LABORATORY MANUAL SECOND EDITION (Sambrook et al., Cold Spring Harbor Laboratory Press), and more specifically, a temperature condition of approximately 30° C. (a temperature condition approximately 5 to 10° C. higher than Tm of the probe sequence), a salt concentration condition of less than 1 M or the like.

When using the solid carrier for detecting mycobacteriosis or mycobacterial infection of the embodiment for miRNA detection, in addition to a probe immobilized onto a solid carrier (hereinafter, also called “immobilized probe”), detection probes may be used. When the detection probe is used, the immobilized probe may include a base sequence that is not complementary to miRNA but can hybridize with the detection probe, in addition to a sequence complementary to miRNA. The detection probe can be labeled with a labeling substance. The location of the labeling substance in the detection probe is not particularly limited as long as it is not binding to the end adjacent to the miRNA.

Examples of the labeling substances include fluorescent dyes, fluorescent beads, quantum dots, biotin, antibodies, antigens, energy absorbing substances, radioisotopes, chemiluminescent materials, and enzymes.

Examples of fluorescent dyes include carboxyfluorescein (FAM), 6-carboxy-4′,5′-dichloro 2′,7′-dimethoxyfluorescein (JOE), fluorescein isothiocyanate (FITC), tetrachlorofluorescein (TET), 5′-hexachloro-fluorescein-CE phosphoramidite (HEX), Cy3, Cy5, Alexa568, and Alexa647.

As described above, since only a trace of miRNA is included in total RNA, it is difficult to label the miRNA with high efficiency. Meanwhile, when a labeled detection probe is used, a trace of miRNA can be detected with high sensitivity.

Further, immobilized probe may be labeled with a labeling substance. In this case, a combination of a labeling substance for labeling the immobilized probe and a labeling substance for labeling a detection probe may be a combination in which fluorescence (Foerster) resonance energy transfer (FRET) cannot occur or a combination in which FRET may occur.

From the viewpoint that FRET efficiency varies depending on the sequence or length of the miRNA, a combination in which FRET cannot occur is preferable. Even when a combination of labeling substances which may cause FRET is used, this can also be designed such that FRET does not occur between both substances, for example, in the manner in which an end of the immobilized probe, which is adjacent to the solid carrier, is labeled with FAM, and the part of the detection probe furthest from the solid carrier is labeled with Alexa647.

On the other hand, from the viewpoint that the case in which the detection probe is linked to the immobilized probe can be distinguished from the case in which the detection probe is attached to the solid carrier, the combination that may cause FRET is preferable.

As a combination of labeling substances in which FRET may occur, a combination of a fluorescent dye with an excitation wavelength of approximately 490 nm (e.g., FITC, Rhodamine green, Alexa™ fluor 488 or BODIPY FL) and a fluorescent dye with an excitation wavelength of approximately 540 nm (e.g., tetramethylrhodamine (TAMRA) or Cy3) or a combination of a fluorescent dye with an excitation wavelength of approximately 540 nm and a fluorescent dye with an excitation wavelength of approximately 630 nm (e.g., Cy5) is an exemplary example.

<<Kit for Detecting Mycobacteriosis or Mycobacterial Infection>>

As an embodiment, the present invention provides a kit for detecting mycobacteriosis or mycobacterial infection, which includes: a reverse transcription primer including a base sequence complementary to a partial sequence at the 3′ end of a base sequence having 85% or more identity with respect to a base sequence set forth in SEQ ID NO: 1; forward and reverse primers for amplifying a reverse transcription product that can be obtained by reverse transcription using miRNA consisting of the base sequence having 85% or more identity with respect to the base sequence set forth in SEQ ID NO: 1 and the above-mentioned reverse transcription primer; and a labeled oligonucleotide probe comprising a sequence complementary to the amplification product obtained by the forward and reverse primers.

The kit for detecting mycobacteriosis or mycobacterial infection of the embodiment can be used to measure the above-described miRNA by quantitative RT-PCR. According to the kit for detecting mycobacteriosis or mycobacterial infection of the embodiment, the activity of mycobacteriosis or the presence or absence of mycobacterial infection can be easily and accurately evaluated.

The reverse transcription primer of the embodiment includes a base sequence complementary to a partial sequence at the 3′ end of a base sequence having 85% or more identity with respect to the base sequence set forth in SEQ ID NO: 1. The partial sequence at the 3′ end can be, for example, 1, 2, 3, 4, or 5 bases from the 3′ end of the target miRNA. The above-mentioned base sequence can be annealed to the target miRNA and extended by reverse transcriptase, thereby synthesizing cDNA.

The reverse transcription primer in the embodiment may include a base sequence capable of forming a loop structure, in addition to the sequence complementary to a partial sequence of the 3′ end of a base sequence having 85% or more identity with respect to the base sequence set forth in SEQ ID NO: 1. When the reverse transcription primer of the embodiment has a loop structure, a polynucleotide in which a reverse transcription primer and cDNA synthesized by the reverse transcriptase are linked may be obtained as a reverse transcription product, and miRNA can be detected with higher specificity by using a reverse primer specific to the base sequence of the loop structure of the reverse transcription primer in subsequent real-time PCR.

The forward primer of the embodiment can be a sequence specific to the base sequence at the 3′ end region of cDNA derived from miRNA consisting of the base sequence having 85% or more identity with respect to the base sequence set forth in SEQ ID NO: 1 of the reverse transcription product obtained with the above-mentioned reverse transcription primer. In addition, the reverse primer of the embodiment can be a sequence specific to a base sequence of the 5′ end region of the reverse transcription primer. Since the forward primer and the reverse primer have the above-described sequences, the reverse transcription product can be more specifically amplified in the real-time PCR.

The labeled oligonucleotide probe of the embodiment includes a sequence complementary to a part of the amplification product by the forward primer and the reverse primer. The oligonucleotide probe may include a base sequence that is the same as at least three bases from the 3′ end of the base sequence having 85% or more identity with respect to the base sequence set forth in SEQ ID NO: 1. The probe including the above-mentioned base sequence can be specifically hybridized with the reverse transcription product. As the labeled probe of the embodiment, any one in which a quencher binds to the 5′ end and a labeling substance binds to the 3′ end can be used. When the primer has a quencher at the 5′ end and a labeling substance at the 3′ end, in the extension performed by a DNA polymerase in the real-time PCR, the probe hybridized with the reverse transcription product can be degraded due to the 5′→3′ exonuclease activity of the polymerase, and thus the suppression by the quencher is canceled and the labeling substance can be detected.

As the labeling substance binding to the 3′ end of the probe, one that is the same as the labeling substance of the detection probe in the above-described <<solid carrier for detecting mycobacteriosis or mycobacterial infection>> may be used, and it can be, for example, a fluorescent dye. When the labeling substance binding to the 3′ end of the probe is a fluorescent dye, as a combination of the labeling substance binding to the 3′ end of the probe and the quencher binding to the 5′ end of the probe, a combination of labeling substances in which FRET between the detection probe and the immobilized probe in the above-described <<solid carrier for detecting mycobacteriosis or mycobacterial infection>> may occur may be used.

Besides the labeling substances, the kit for detecting mycobacteriosis or mycobacterial infection of the embodiment may include one or more of a nucleotide triphosphate as a substrate, a nucleic acid polymerase and a buffer for amplification according to the method for amplifying the nucleic acid used herein. The nucleotide triphosphate is a substrate (dNTP or rNTP) according to a nucleic acid polymerase. The nucleic acid polymerase is an enzyme according to the method for amplifying the nucleic acid used herein, which is, for example, DNA polymerase, RNA polymerase, or reverse transcriptase. As the buffer for amplification, Tris buffer, phosphate buffer, Veronal buffer, boric acid buffer or Good's buffer may be used, and there is no particular limitation to pH.

The present invention will be described with reference to examples below, but the present invention is not limited thereto.

### EXAMPLES

### Example 1

(1) Selection of Specimens

Sixteen for each group of untreated female patients diagnosed with MAC pulmonary disease (patient group: MAC) and female healthy subjects (healthy subject group: CON) were selected, and it was confirmed that there was no statistical difference between the two groups, and that the groups were matched for ages and BMI.

(2) Extraction of miRNA from Serum

Blood was taken from the MAC group or the CON group selected in (1) to obtain serum. From the resulting serum, miRNA was extracted using a NucleoSpin™ miRNA plasma (manufactured by Macherey-Nagel). As an extrinsic control, cel-miR-39 (100 fmol) was added. From 300 μL of the serum, 35 μL of miRNA extract eluted with RNase-free water was obtained.

(3) Synthesis of cDNA

Complementary DNA (cDNA) was synthesized by reverse transcription using the miRNA extract obtained in (2) and a TaqMan™ MicroRNA reverse Transcription Kit (manufactured by Applied Biosystems by ThermoFisher Scientific). Specifically, for the reaction, a total of 15 μL of a reaction solution including 3 μL of the miRNA extract, 1× stem loop reverse transcription primer (included in TaqMan™ MicroRNA Assays; manufactured by Applied Biosystems by ThermoFisher Scientific), 3.33 U/μL of a reverse transcriptase, 0.25 U/μL of an RNase inhibitor, 0.25 mM of dNTPs and 1× reaction buffer was prepared, and then reacted using a Thermal Cycler-Gene Amp PCR System 9700 (manufactured by Applied Biosystems by ThermoFisher Scientific) at 16° C. for 30 minutes, 42° C. for 30 minutes, and 85° C. for 5 minutes.

(4) Real-Time PCR

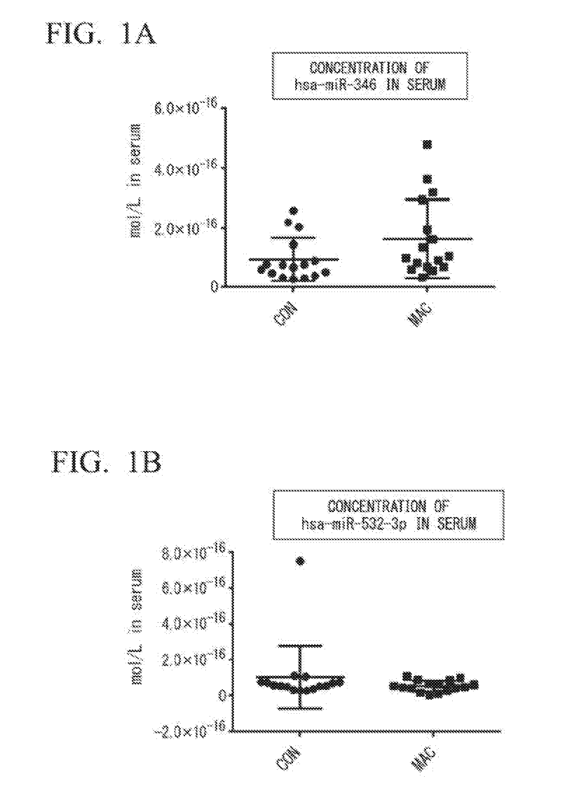

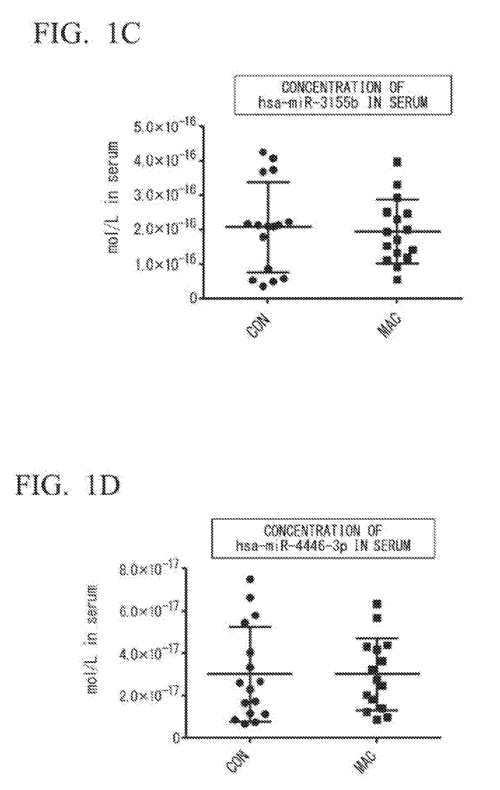

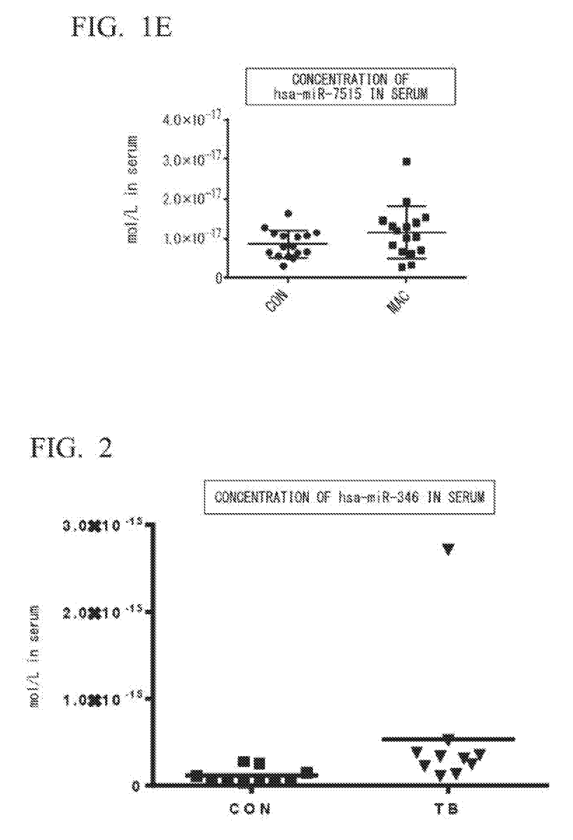

By using a TaqMan™ probe, hsa-miR-346 was quantified. Specifically, 1 μL of TaqMan™ MicroRNA Assays (manufactured by Applied Biosystems by ThermoFisher Scientific; containing 20× forward primer, 20× reverse primer and 20× probe), and 10 μL of TaqMan™ Universal Master Mix II (containing UNG; manufactured by Applied Biosystems by ThermoFisher Scientific) were added to 1.33 μL of the reverse transcription reaction solution obtained in (3), thereby preparing a total of 20 μL of a quantitative reaction solution. Subsequently, the resulting solution was heated using a StepOnePlus™ real-time PCR system (manufactured by Applied Biosystems by ThermoFisher Scientific) at 50° C. for 2 minutes and at 95° C. for 10 minutes, followed by real-time PCR for 45 cycles of 95° C. for 15 seconds and 60° C. for 60 seconds. Each reaction was measured twice, and for each measurement, a calibration curve was plotted using a miRNA mimic (Bioneer), and to correct variations between the experiments, an amount of cel-miR-39 added as an extrinsic control and an amount of intrinsic hsa-miR-16-5p that is constant in expression level in serum were measured. The results are shown in FIGS. 1A to 1E. FIGS. 1A to 1E show miRNA concentrations in serum of 5 types of candidate for a biomarker of mycobacteriosis selected by comprehensive analysis conducted in advance.

In FIGS. 1A to 1E, it was noted that, in the MAC group, the concentration of hsa-miR-346 in serum among the 5 types of miRNA was higher than that of the healthy group, which had a statistical significance (p<0.05).

### Example 2

(1) Selection of Specimens

Ten for each group of untreated patients diagnosed with tuberculosis (patient group: TB) and healthy subjects (healthy subject group: CON) were selected, and it was confirmed that there was no statistical difference between the two groups, and that the groups were matched for ages and BMI.

(2) Extraction of miRNA from Serum

Blood was taken from the MAC group or the CON group selected in (1) to obtain serum. From the resulting serum, an miRNA extract was obtained by the method as described in Example 1(2).

(3) Synthesis of cDNA

Complementary DNA (cDNA) was synthesized using the miRNA extract obtained in (2) and the method as described in Example 1(3).

(4) Real-Time PCR

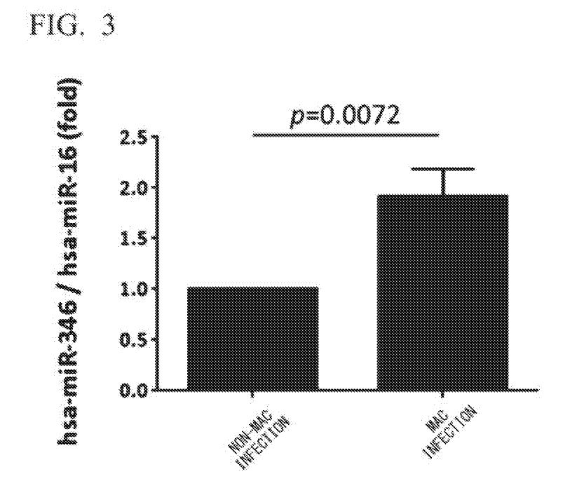

By using 1.33 μL of the reverse transcription reaction solution obtained in (3), hsa-miR-346 was quantified by a method similar to that described in Example 1(4). The results are shown in FIG. 2.

FIG. 2 shows that, in the TB group, the concentration of hsa-miR-346 in serum was higher than that of the CON group, with a statistical significance (p<0.0015).

### Example 3

(1) Cell Culture

Human peripheral blood mononuclear cell (PBMC)-derived macrophages were cultured to confluence in a 6-well plate. As a medium, Iscove's Modified Dulbecco's Media (IMDM) (manufactured by ThermoFisher Scientific Co., Ltd.) was used.

(2) MAC Infection

The macrophages cultured in (1) were infected with MAC104 at a multiplicity of infection (MOI) of 50, and cultured at 37° C. for 4 hours. As a control, non-infected cells were prepared.

(3) Quantification of miRNA

Subsequently, the cells were washed with PBS three times, the medium was replaced with 600 μL of IMDM (serum-free), and the cells were cultured at 37° C. therein. After 24 hours, quantification of hsa-miR-346 in 300 μL of a cell supernatant was carried out. For quantification, miRNA extraction, cDNA synthesis and real-time PCR were carried out by the methods described in Examples 1(2) to (4). The results are shown in FIG. 3.

In FIG. 3, it was seen that, in MAC-infected cells, the concentration of hsa-miR-346 in serum was statistically significantly higher than that in the macrophages not infected by MAC.

From the above-described results, it was noted that miR-346 is useful as a biomarker of mycobacteriosis.

### Example 4

(1) Selection of Specimens

To determine whether the concentration of hsa-miR-346 in serum reflects the activity of MAC pulmonary disease, the results of culturing sputum for two female patients with MAC pulmonary disease and the change in concentration of hsa-miR-346 in serum over time from the initiation of treatment were analyzed. These patients were admitted to Keio University Hospital from July 2013 to June 2016, met the official statement on NTM disease established by American Thoracic Society (ATS) and the Infections Society of America (IDSA), and received combination chemotherapy including clarithromycin (CLM).

(2) Extraction of miRNA from Serum

Blood was taken from the two patients with MAC pulmonary disease selected from (1) and healthy volunteers with consent to participate in research, and then serum was obtained therefrom. From the serum, an miRNA extract was obtained by the method as described in Example 1(2). In addition, the research was conducted in compliance with the Helsinki Declaration, and approved by the Institutional Ethics Review Committee of Human Studies in Keio University School of Medicine and Hospital (20130134).

(3) Synthesis of cDNA

Complementary DNA (cDNA) was synthesized using the miRNA extract obtained in (2) and the method as described in Example 1(3).

(4) Real-Time PCR

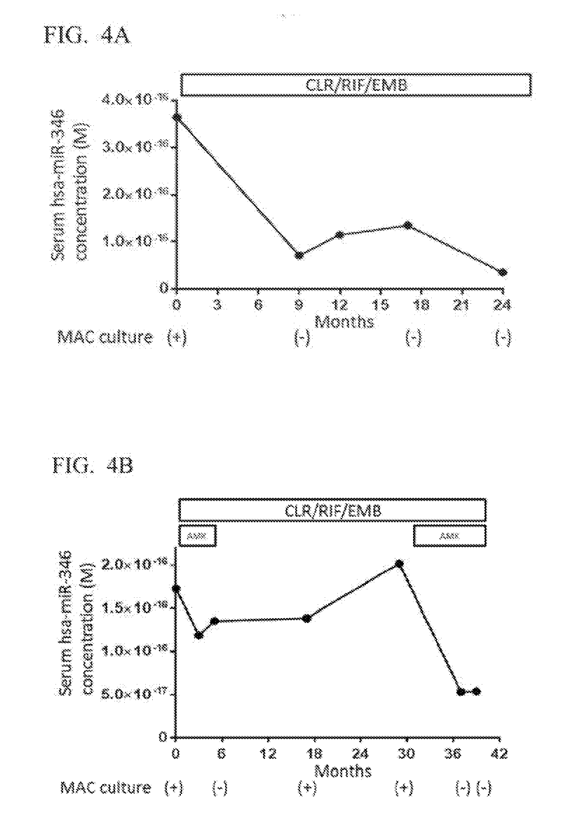

By using the reverse transcription reaction solution obtained in (3), hsa-miR-346 was quantified by a method similar to Example 1(4). The results are shown in FIGS. 4A and 4B.

FIG. 4A shows a measurement result for a 55-year-old woman with a small amount of sputum who came to Keio University Hospital. She had no medical history, and had not received treatment for mycobacteriosis. In addition, the patient was diagnosed with nodular bronchiectatic MAC pulmonary disease. Subsequently, treatment for mycobacteriosis using antibiotics (600 mg/day of rifampicin (RIF), 750 mg/day of ethambutol (EMB), and 800 mg/day of clarithromycin (CLN)) was started.

In FIG. 4A, the sputum culture results change from positive to negative 6 months after the treatment, and the concentration of hsa-miR-346 in serum decreased.

FIG. 4B shows measurement results for a 66-year-old woman with moist cough, who came to Keio University Hospital. The patient received treatment for hyperlipidemia, but did not receive treatment for mycobacteriosis. In addition, the patient was diagnosed with nodular bronchiectatic MAC pulmonary disease. Subsequently, treatment for mycobacteriosis using four types of antibiotic (600 mg/day of rifampicin (RIF), 750 mg/day of ethambutol (EMB), 800 mg/day of clarithromycin (CLN), and 600 mg of amikacin (AMK) every other day) was started.

In FIG. 4B, the sputum culture result changed from positive to negative 4 months after the start of treatment, and the concentration of hsa-miR-346 in serum decreased.

In addition, when the treatment with AMK was stopped four months after the start of treatment, the sputum culture result changed from negative to positive 10 months after the start of treatment, and the concentration of hsa-miR-346 in serum increased.

Further, when AMK was administered again 32 months after the start of treatment, the sputum culture result changed from positive to negative after three months of the additional AMK administration. In addition, the concentration of hsa-miR-346 in serum decreased together with the change in the sputum culture result.

From the above results, it was confirmed that the concentration of hsa-miR-346 in serum is associated with the disease activity determined by the sputum culture result.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

### SEQUENCE LISTING

<110> NIKON CORPORATION
   KEIO UNIVERSITY
<120> Biomarker of acid-fast bacilli disease or acid-fast bacilli infection
<130> OSP68447
<160> 1
<170> PatentIn version 3.5
<210> 1
<211> 23
<212> RNA
<213> Homo sapiens
<400>1

| | |
|---|---|
| ugucugcccg caugccugcc ucu | 23 |

What is claimed is:

1. A solid carrier for detecting mycobacteriosis or mycobacterial infection, comprising:
at least one probe including a polynucleotide consisting of a base sequence complementary to all or a part of a base sequence having 85% or more identity with respect to a base sequence set forth in SEQ ID NO: 1, which is immobilized on the surface of the solid carrier.

2. The solid carrier according to claim 1, which is used for evaluating the activity of mycobacteriosis or the presence or absence of the activity of mycobacterial infection.

3. A method for evaluating the activity of mycobacteriosis or the presence or absence of mycobacterial infection, comprising:
bringing a sample derived from a subject into contact with a solid carrier which detects mycobacteriosis or mycobacterial infection comprising at least one probe including a polynucleotide consisting of a base sequence complementary to all or a part of a base sequence having 85% or more identity with respect to a base sequence set forth in SEQ ID NO: 1, which is immobilized on the surface of the solid carrier; and
detecting a nucleic acid hybridized with the probe.

4. The method according to claim 3, wherein the subject is suspected to have mycobacteriosis or to be infected with mycobacteria.

5. The method according to claim 3, wherein the subject has mycobacteriosis or is injected with mycobacteria.

6. The method according to claim 5, wherein the subject receives treatment for mycobacteriosis.

7. A kit for detecting mycobacteriosis or mycobacterial infection, comprising:
a reverse transcription primer including a base sequence complementary to a partial sequence at the 3′ end of a base sequence having 85% or more identity with respect to a base sequence set forth in SEQ ID NO: 1;
forward and reverse primers for amplifying a reverse transcription product that is obtained by reverse transcription using miRNA consisting of a base sequence having 85% or more identity with respect to the base sequence set forth in SEQ ID NO: 1 and the reverse transcription primer; and
a labeled oligonucleotide probe comprising a sequence complementary to a part of the resulting amplification product obtained by the forward and reverse primers.

8. The kit according to claim 7, which is used for evaluating the activity of mycobacteriosis or the presence or absence of mycobacterial infection.

9. The kit according to claim 7, wherein the oligonucleotide probe includes a base sequence identical to at least three bases on the 3′ end of the base sequence having 85% or more identity with respect to the base sequence set forth in SEQ ID NO: 1.

10. The kit according to claim 7, wherein the reverse transcription primer includes a base sequence capable of forming a loop structure, in addition to the base sequence complementary to a partial sequence of the 3′ end of the base sequence having 85% or more identity with respect to the base sequence set forth in SEQ ID NO: 1.


SEQUENCE LISTING (1 sequence)

SEQ ID NO: 1 - RNA, 23 nt, Homo sapiens
ugucugcccgcaugccugccucu